(19) United States
(12) Patent Application Publication
(10) Pub. No.: US 2011/0015635 A1
(43) Pub. Date: Jan. 20, 2011

(54) APPARATUS AND METHOD FOR FACILITATING INTERVERTEBRAL ARTHRODESIS AND DISC SPACE PREPARATION AS PART OF TREATMENT OF SPINAL DEGENERATIVE DISEASE

(76) Inventor: Henry E. Aryan, Fresno, CA (US)

Correspondence Address: GREGORY SMITH & ASSOCIATES, 3900 NEWPARK MALL ROAD, 3RD FLOOR, NEWARK, CA, 94560, US

(21) Appl. No.: 12/838,392

(22) Filed: Jul. 16, 2010

Related U.S. Application Data
(60) Provisional application No. 61/226,595, filed on Jul. 17, 2009.

(51) Int. Cl. A61B 17/16

(52) U.S. Cl. 606/84

(57) ABSTRACT

The apparatus of the invention is an adjustable intervertebral shaver with a handle portion and a blade portion that adjusts width while remaining in the disc space. The invention also includes methods using the adjustable intervertebral shaver to surgically prepare the intervertebral space. By changing the width of the blade portion, the surgeon adjusts the aggressiveness of the curetting and also determines the appropriate size for an intervertebral graft. By remaining in the disc space, this decreases the time it may take to appropriately prepare the disc space. In addition, since the blade portion remains in the disc space it reduces the number of times an instrument must be passed adjacent to nerves and neural elements, thus decreasing the likelihood of damage to these structures. The width of the blade portion adjusts via an adjustment knob on the handle portion of the adjustable intervertebral shaver.

### CROSS REFERENCE TO OTHER APPLICATIONS

This application claims the benefit of U.S. Provisional Application number 61/226,595, filed on Jul. 17, 2009, which is incorporated by reference in its entirety.

### TECHNICAL FIELD

The invention relates to apparatus and methods for preparation of the intervertebral disc space as performed during surgery to fuse vertebrae. Vertebrae are fused in an attempt to alleviate pain and restore neurologic function associated with disease that affects the cervical, thoracic, and lumbar discs and their adjacent vertebra. The technology also applies to all circumstances in which intervertebral bony arthrodesis may be desired.

### BACKGROUND OF THE INVENTION

Degenerative disc disease is a condition related to the degenerative of intervertebral discs and the adjacent vertebrae. These discs normally serve as cushions between vertebrae and allow for some degree of motion of the vertebrae. In the setting of degeneration, bone spurs may form which may be the source of pain. Further, disc material may herniate into the spinal canal causing neurologic injury. A common treatment for degenerative disc disease is to remove the degenerated disc and/or bone spurs and to fuse the vertebra together. This is often done with the use of instrumentation, namely titanium screws/plates to hold the vertebra in correct position until the fusion takes place. Bone is usually placed in the intervertebral space after removing the disc. Often synthetic intervertebral spaces, or cages, are used to restore disc height and allow a space for fusion to occur. These cages are commonly made of titanium, stainless steel, polyetheretherketone (PEEK), graphite, or carbon fiber. In order to prepare the intervertebral space for arthrodesis, the intervertebral contents must be removed. This is often done with the assistance of curettes, shavers, rongeurs, high-speed drill, and other tissue removing devices. This device facilitates the preparation of the disc space for arthrodesis (spinal fusion) or for arthroplasty (placement of an artificial disc).

### SUMMARY OF THE INVENTION

The apparatus of the invention is an adjustable intervertebral or interbody shaver with a handle portion and a blade portion that adjusts width while remaining in the disc space. The invention also includes methods using the adjustable intervertebral shaver to surgically prepare the intervertebral space. By changing the width of the blade portion, the surgeon adjusts the aggressiveness of the curetting and also determines the appropriate size for an intervertebral graft. By remaining in the disc space, this decreases the time it may take to appropriately prepare the disc space. In addition, since the shaver remains in the disc space it reduces the number of times an instrument must be passed adjacent to nerves and neural elements, thus decreasing the likelihood of damage to these structures. The width of the blade portion adjusts via an adjustment knob on the handle portion of the adjustable intervertebral shaver.

### BRIEF DESCRIPTION OF THE DRAWINGS

FIG. 1 is a side view of the adjustable intervertebral shaver of the present invention shown in a contracted or unexpanded position.

FIG. 2 is an end view of the adjustable intervertebral shaver of FIG. 1 in the contracted or unexpanded position.

FIG. 3 is a side view of the adjustable intervertebral shaver of FIG. 1 shown in an expanded position.

FIG. 4 is an end view of the adjustable intervertebral shaver of FIG. 3 in the expanded position.

FIG. 5 illustrates the adjustable intervertebral shaver of FIGS. 1-4 in use for surgically preparing an intervertebral space.

FIG. 6 is a cut-away side view of an adjustable intervertebral shaver with a screw-type expansion mechanism shown in an unexpanded position.

FIG. 7 is a cut-away end view of the adjustable intervertebral shaver of FIG. 6 in the unexpanded position.

FIG. 8 is a cut-away side view of the adjustable intervertebral shaver of FIG. 6 in an expanded position.

FIG. 9 is a cut-away end view of the adjustable intervertebral shaver of FIG. 8 in the expanded position.

FIG. 10 is a cut-away side view of an adjustable intervertebral shaver with a scissor-type expansion mechanism shown in an unexpanded position.

FIG. 11 is a cut-away end view of the adjustable intervertebral shaver of FIG. 10 in the unexpanded position.

FIG. 12 is a cut-away side view of the adjustable intervertebral shaver of FIG. 10 in an expanded position.

FIG. 13 is a cut-away end view of the adjustable intervertebral shaver of FIG. 12 in the expanded position.

FIG. 14 is a cut-away side view of another adjustable intervertebral shaver with a scissor-type expansion mechanism shown in an unexpanded position.

FIG. 15 is a cut-away end view of the adjustable intervertebral shaver of FIG. 14 in the unexpanded position.

FIG. 16 is a cut-away side view of the adjustable intervertebral shaver of FIG. 14 in an expanded position.

FIG. 17 is a cut-away end view of the adjustable intervertebral shaver of FIG. 16 in the expanded position.

FIG. 18 is a cut-away side view of an adjustable intervertebral shaver with a rack-and-pinion expansion mechanism shown in an unexpanded position.

FIG. 19 is a cut-away end view of the adjustable intervertebral shaver of FIG. 18 in the unexpanded position.

FIG. 20 is a cut-away side view of the adjustable intervertebral shaver of FIG. 18 in an expanded position.

FIG. 21 is a cut-away end view of the adjustable intervertebral shaver of FIG. 20 in the expanded position.

FIG. 22 is a cut-away side view of another adjustable intervertebral shaver with a rack-and-pinion expansion mechanism shown in an unexpanded position.

FIG. 23 is a cut-away end view of the adjustable intervertebral shaver of FIG. 21 in the unexpanded position.

FIG. 24 is a cut-away side view of the adjustable intervertebral shaver of FIG. 21 in an expanded position.

FIG. 25 is a cut-away end view of the adjustable intervertebral shaver of FIG. 23 in the expanded position.

### DETAILED DESCRIPTION OF THE INVENTION

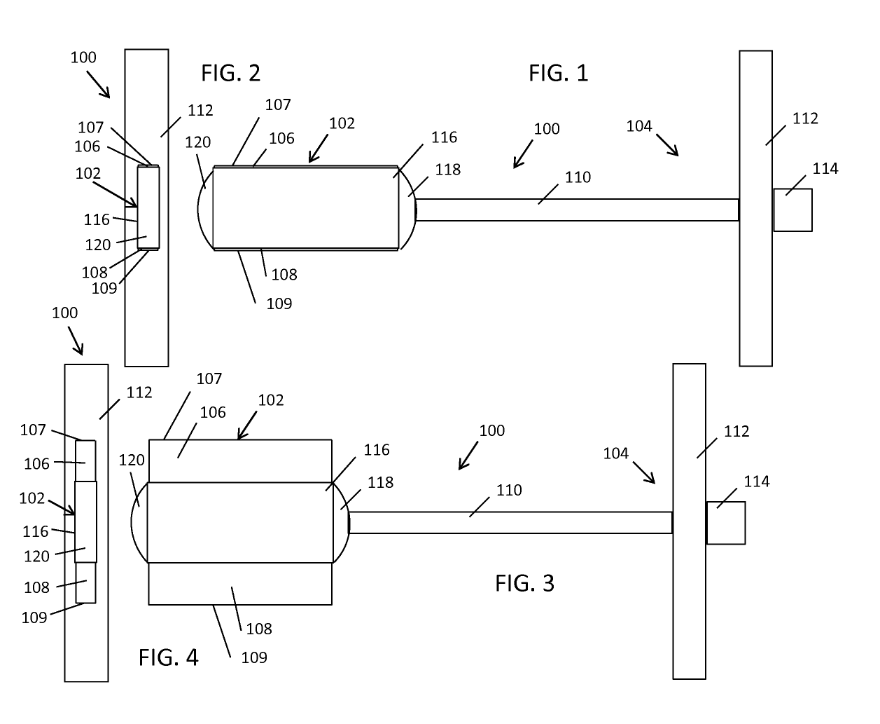

FIG. 1 is a side view and FIG. 2 is an end view of the adjustable intervertebral shaver 100 of the present invention shown in a contracted or unexpanded position. FIG. 3 is a side view and FIG. 4 is an end view of the adjustable intervertebral shaver of FIG. 1 shown in an expanded position. The intervertebral shaver 100 has a distal blade portion 102 that is connected to a proximal handle portion 104. Preferably, the blade portion 102 has a flat, paddle-shaped configuration with a thickness that is generally less than its width. The width of the blade portion 102 is incrementally adjustable from the contracted or unexpanded position (FIGS. 1-2) to the expanded position (FIGS. 3-4). In the embodiment shown in FIGS. 1-4, the blade portion 102 includes a pair of wings 106, 108 that extend and retract from the lateral sides of a central housing 116 of the blade portion 102 in response to an expansion mechanism enclosed within the intervertebral shaver 100. The lateral edges 107, 109 of the wings 106, 108 provide scraping or cutting surfaces that may be configured as straight or convex surfaces. Other geometries of scraping or cutting surfaces known in the industry may also be applied to the lateral edges 107, 109 of the wings 106, 108. Preferably, the width of the blade portion 102 will have an expansion ratio of approximately 2 times from a fully contracted position to a fully expanded position, however different expansion mechanisms may provide different expansion ratios. Preferably, the width of the blade portion 102 will be incrementally adjustable from approximately 6-7 mm up to approximately 12-14 mm. The thickness of the blade portion 102 will preferably be approximately 1-3 mm, most preferably approximately 1.5 mm. The length of the blade portion 102 will preferably be approximately 20-40 mm.

The handle portion 104 includes a shaft 110 and a T-handle 112 that are connected to the blade portion 102. The T-handle 112 is preferably aligned with the width of the blade portion 102 to provide a visual indication of the position of the blade portion 102 during use. The length of the shaft 110 will typically be approximately 25-35 cm. The typically of the T-handle 112 will preferably be approximately 10-12 cm. An adjustment knob 114 is provided on the handle portion 104 for operating the expansion mechanism for the blade portion 102. Preferably, the adjustment knob 114 will be marked to provide a visual indication of the width that the blade portion 102 has been adjusted to.

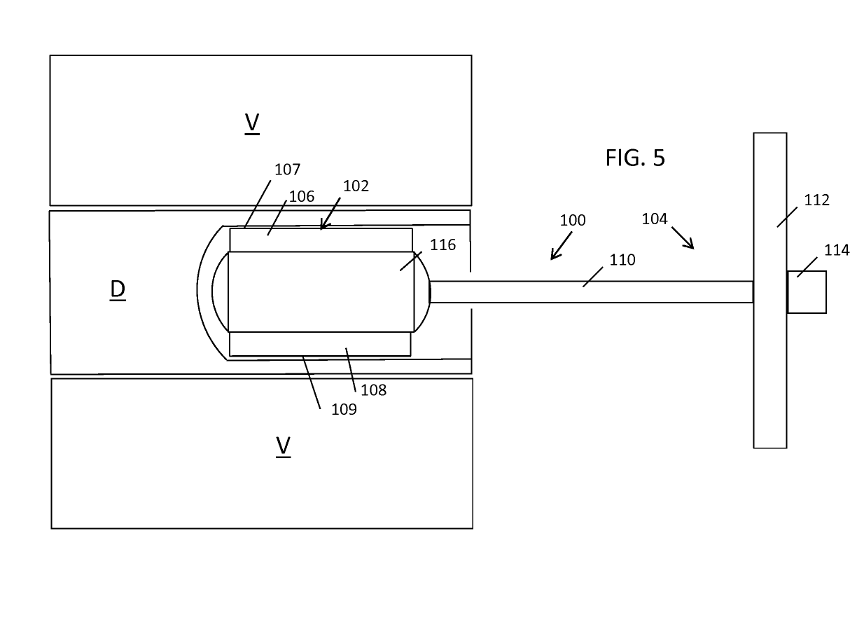

FIG. 5 illustrates the adjustable intervertebral shaver 100 of FIGS. 1-4 in use for surgically preparing an intervertebral space prior to arthrodesis or arthroplasty. The blade portion 102 of the intervertebral shaver 100 is adjusted to its smallest size and then inserted in a flat orientation into the intervertebral space between two vertebral bodies V after an incision is made in the disc D with a knife or other instrument. Using the adjustment knob 114, the blade portion 102 of the intervertebral shaver 100 is adjusted to the desired width and the intervertebral shaver 100 is rotated or oscillated (either clockwise and/or counterclockwise) with the T-handle 112 to remove disc material and scrape the cartilaginous vertebral endplates. If desired, the width of the blade portion 102 can be increased without removing it from the disc space and the process can be repeated until a sufficient amount of disc material has been broken up. Typically, these steps are repeated 3-4 times. The blade portion 102 of the intervertebral shaver 100 is then collapsed to its smallest size and withdrawn through the incision. The broken up disc material can be removed from the disc D with the assistance of rongeurs, suction devices or other instruments.

The adjustable width of the intervertebral shaver 100 allows it to do the work of several different sizes of standard intervertebral shavers without the need for repeated insertions and withdrawals of multiple instruments. This will reduce the time needed for preparing the intervertebral space and it will improve the safety of the procedure because fewer instruments will have to be inserted or withdrawn past the nerves and other sensitive structures close to the spine. If necessary, the intervertebral shaver 100 can be reinserted into the disc D and the process repeated. Even if the intervertebral shaver 100 must be reinserted, the fact that it can be contracted to a smaller size before insertion or withdrawal will improve the safety of the procedure.

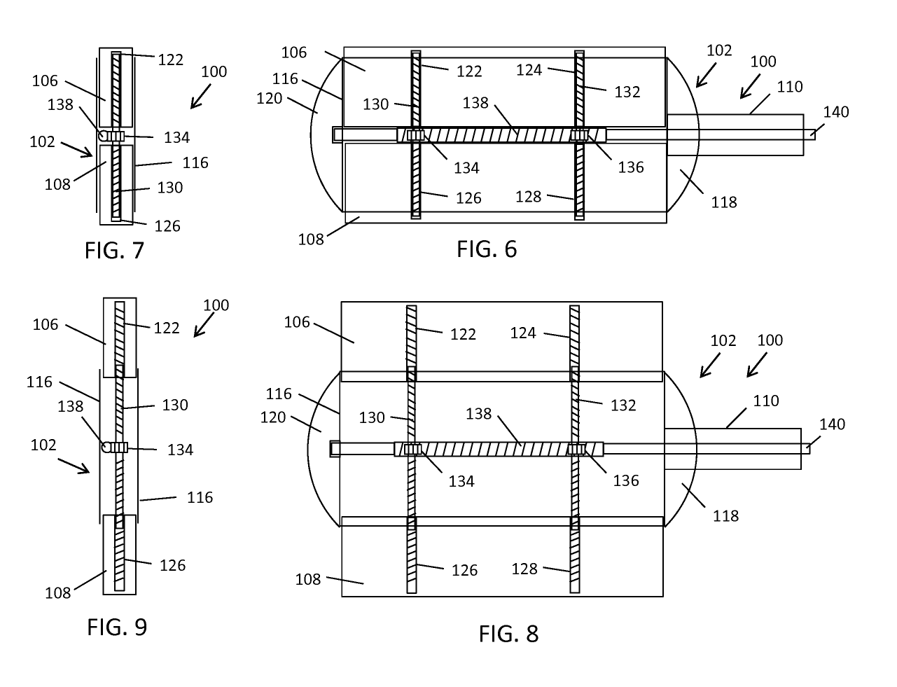

Various expansion mechanisms can be used for extending and retracting the wings 106, 108 of the blade portion 102 of the intervertebral shaver 100. FIG. 6 is a cut-away side view and FIG. 7 is a cut-away end view of an adjustable intervertebral shaver 100 with a screw-type expansion mechanism shown in an unexpanded position. FIG. 8 is a cut-away side view and FIG. 9 is a cut-away end view of the adjustable intervertebral shaver 100 of FIG. 6 in an expanded position.

The wings 106, 108 are configured for a sliding fit within a central housing 116 of the blade portion 102. Optionally, the central housing 116 may be contoured (e.g. rounded or tapered) on the proximal 118 and distal 120 ends of the blade portion 102. A pair of threaded holes 122 & 124, 126 & 128, are located in each of the wings 106, 108. The holes 122 & 124 in the first wing 106 are threaded in the opposite direction from the holes 126 & 128 in the second wing 108. Two threaded rods 130, 132 engage the threaded holes 122 & 124, 126 & 128, in each of the wings 106, 108. The threaded rods 130, 132 are threaded in opposite directions on opposite ends of the threaded rods 130, 132. A pinion gear 134, 136 is attached at the center of each of the threaded rods 130, 132. A worm gear 138 attached to a rotating inner shaft 140 engages each of the pinion gears 134, 136. One long worm gear 138 can be used, as shown, or, alternatively, a pair of smaller worm gears may be used. The inner shaft 140 is connected to the adjustment knob 114 on the handle portion 104 (See FIG. 1).

When the adjustment knob 114 is rotated in a first direction, the inner shaft 140 rotates the worm gear 138, which causes of each of the pinion gears 134, 136 and therefore the threaded rods 130, 132 to rotate, moving the wings 106, 108 apart to increase the width of the blade portion 102, as shown in FIGS. 8 and 9. When the adjustment knob 114 is rotated in the opposite direction, the inner shaft 140 rotates the worm gear 138, which causes of each of the pinion gears 134, 136 and therefore the threaded rods 130, 132 to rotate in the opposite direction, moving the wings 106, 108 together to decrease the width of the blade portion 102, as shown in FIGS. 6 and 7. Stops may be provided to prevent the wings 106, 108 from overextending and becoming disengaged from the threaded rods 130, 132.

The embodiment of the intervertebral shaver 100 shown in FIGS. 6-9 utilizes two threaded rods 130, 132 to assure stable and parallel motion of the wings 106, 108. The same could also be accomplished using a single, centrally positioned threaded rod and a pair of guide rods or tracks to assure stable and parallel motion of the wings 106, 108.

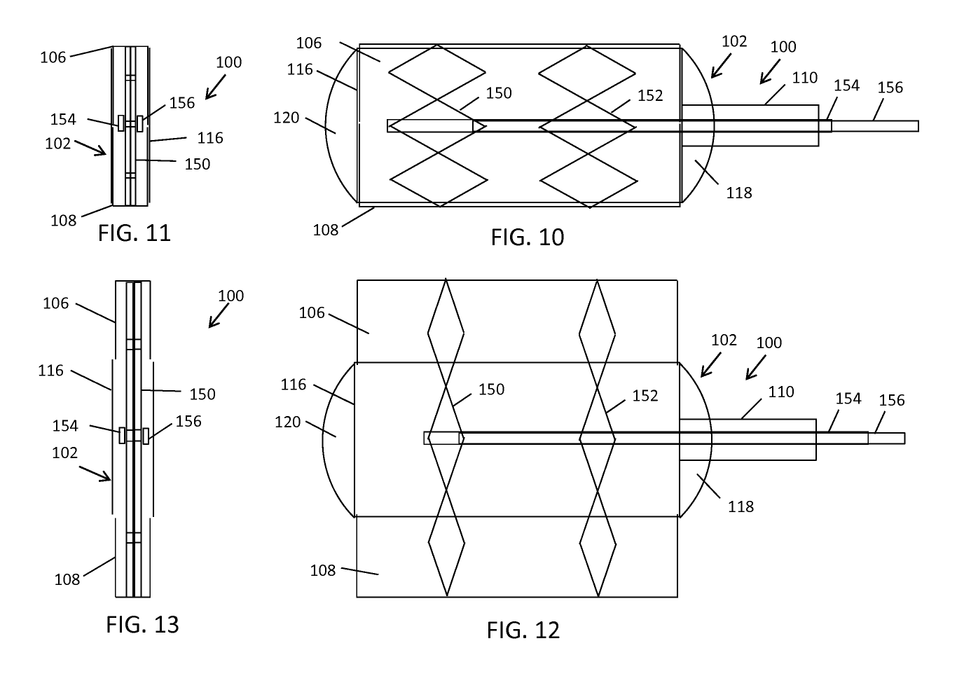

FIG. 10 is a cut-away side view and FIG. 11 is a cut-away end view of an adjustable intervertebral shaver 100 with a scissor-type expansion mechanism shown in an unexpanded position. FIG. 12 is a cut-away side view and FIG. 13 is a cut-away end view of the adjustable intervertebral shaver 100 of FIG. 10 in an expanded position. The wings 106, 108 are configured for a sliding fit within a central housing 116 of the blade portion 102. In this embodiment, the wings 106, 108 of the blade portion 102 are constructed with a hollow configuration to allow additional room for the components of the expansion mechanism. A pair of scissor mechanisms 150, 152 are connected between the first and second wings 106, 108. Each of the scissor mechanisms 150, 152 is made up of eight bars that are pivotally connected to form three diamond-shaped units. The pivot points between the bars can be provided by hinge pins, by flexible, living hinges or by a combination of the two. Other known configurations of scissor mechanisms may also be used.

The opposite sides of the central diamond-shaped unit are connected a pair of push/pull rods 154, 156. When the push/pull rods 154, 156 are moved to decrease the proximal-to-distal length of the central diamond-shaped units in the scissor mechanisms 150, 152, the lateral width of the scissor mechanisms 150, 152 increases, moving the wings 106, 108 apart to increase the width of the blade portion 102, as shown in FIGS. 12 and 13. When the push/pull rods 154, 156 are moved to increase the proximal-to-distal length of the central diamond-shaped units in the scissor mechanisms 150, 152, the lateral width of the scissor mechanisms 150, 152 decreases, moving the wings 106, 108 together to decrease the width of the blade portion 102, as shown in FIGS. 10 and 11. The adjustment knob 114 may actuate the push/pull rods 154, 156 through screw threads, cams, levers or other mechanisms. Stops may be provided to prevent the wings 106, 108 from overextending.

The embodiment of the intervertebral shaver 100 shown in FIGS. 10-13 utilizes two scissor mechanisms 150, 152 to assure stable and parallel motion of the wings 106, 108. The same could also be accomplished using a single, centrally positioned scissor mechanism and a pair of guide rods or tracks to assure stable and parallel motion of the wings 106, 108.

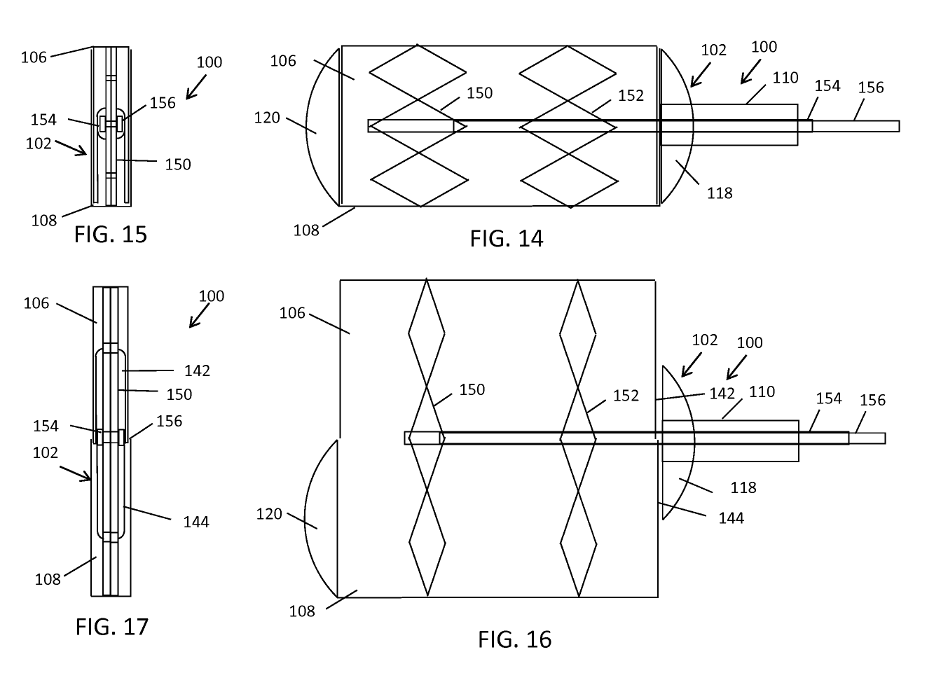

FIG. 14 is a cut-away side view and FIG. 15 is a cut-away end view of another adjustable intervertebral shaver 100 with a scissor-type expansion mechanism shown in an unexpanded position. FIG. 16 is a cut-away side view and FIG. 17 is a cut-away end view of the adjustable intervertebral shaver 100 of FIG. 14 in an expanded position. The scissor-type expansion mechanism operates similarly to the mechanism described above in connection with FIGS. 10-13, however the blade portion 102 of the intervertebral shaver 100 is configured differently. There is no central housing, instead the first wing 106 is configured to slide telescopically within the second wing 108. Slots or openings 142, 144 in the proximal faces of the wings 106, 108 are provided for the push/pull rods 154, 156. The slots 142, 144 are covered by a contoured proximal part 118 of the blade portion 102 so that the interior of the blade portion 102 and the expansion mechanism will not be open to tissue or bodily fluids. Optionally, a contoured distal part 120 of the blade portion 102 may be attached to the second wing 108.

As in FIGS. 10-13, when the push/pull rods 154, 156 are moved to decrease the proximal-to-distal length of the central diamond-shaped units in the scissor mechanisms 150, 152, the lateral width of the scissor mechanisms 150, 152 increases, telescopically sliding the wings 106, 108 apart to increase the width of the blade portion 102, as shown in FIGS. 16 and 17. When the push/pull rods 154, 156 are moved to increase the proximal-to-distal length of the central diamond-shaped units in the scissor mechanisms 150, 152, the lateral width of the scissor mechanisms 150, 152 decreases, telescopically sliding the wings 106, 108 together to decrease the width of the blade portion 102, as shown in FIGS. 14 and 15. Stops may be provided to prevent the wings 106, 108 from overextending.

The embodiment of the intervertebral shaver 100 shown in FIGS. 14-17 also utilizes two scissor mechanisms 150, 152 to assure stable and parallel motion of the wings 106, 108. The same could also be accomplished using a single, centrally positioned scissor mechanism and a pair of guide rods or tracks to assure stable and parallel motion of the wings 106, 108.

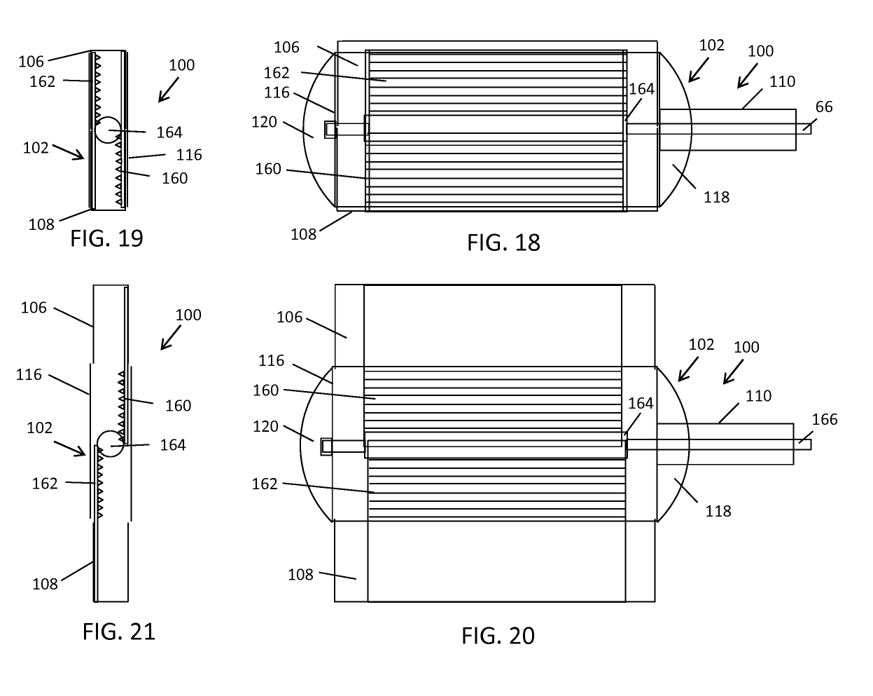

FIG. 18 is a cut-away side view and FIG. 19 is a cut-away end view of an adjustable intervertebral shaver 100 with a rack-and-pinion expansion mechanism shown in an unexpanded position. FIG. 20 is a cut-away side view and FIG. 21 is a cut-away end view of the adjustable intervertebral shaver of FIG. 18 in an expanded position. The wings 106, 108 are configured for a sliding fit within a central housing 116 of the blade portion 102. In this embodiment, the wings 106, 108 may be hollow or solid, as desired. A rack-type gear 160, 162 is attached to each of the wings 106, 108. Each rack gear 160, 162 is engaged by a pinion gear 164 on a rotating inner shaft 166. The inner shaft 166 is connected to the adjustment knob 114 on the handle portion 104 (See FIG. 1).

When the adjustment knob 114 is rotated in a first direction, the inner shaft 166 rotates the pinion gear 164, which causes of each of the rack gears 160, 162 and therefore the wings 106, 108 to move outward to increase the width of the blade portion 102, as shown in FIGS. 20 and 21. When the adjustment knob 114 is rotated in the opposite direction, the inner shaft 166 rotates the pinion gear 164 in the opposite direction, which causes of each of the rack gears 160, 162 and therefore the wings 106, 108 to move inward to decrease the width of the blade portion 102, as shown in FIGS. 18 and 19. Stops may be provided to prevent the wings 106, 108 from overextending and to keep the rack gears 160, 162 from becoming disengaged from the pinion gear 164.

The embodiment of the intervertebral shaver 100 shown in FIGS. 18-21 utilizes a very wide rack-and-pinion gearset to assure stable and parallel motion of the wings 106, 108. The same could also be accomplished using a pair of narrower, spaced apart rack-and-pinion gearsets that act in synchrony to assure stable and parallel motion of the wings 106, 108.

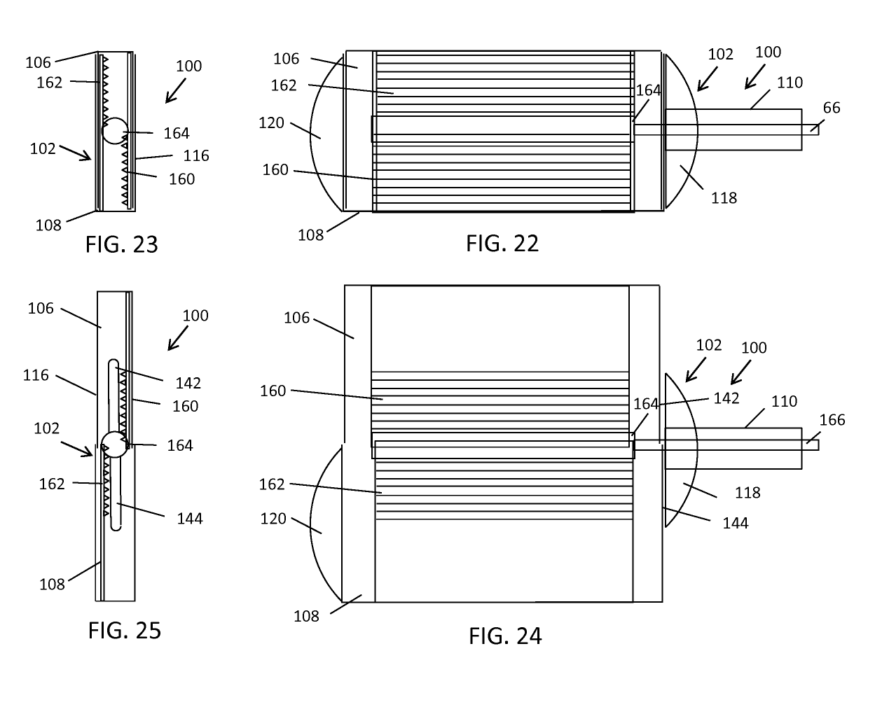

FIG. 22 is a cut-away side view and FIG. 23 is a cut-away end view of another adjustable intervertebral shaver with a rack-and-pinion expansion mechanism shown in an unexpanded position. FIG. 24 is a cut-away side view and FIG. 25 is a cut-away end view of the adjustable intervertebral shaver of FIG. 22 in an expanded position. The rack-and-pinion expansion mechanism operates similarly to the mechanism described above in connection with FIGS. 18-21, however, the blade portion 102 of the intervertebral shaver 100 is configured differently. There is no central housing, instead the first wing 106 is configured to slide telescopically within the second wing 108. Slots or openings 142, 144 in the proximal faces of the wings 106, 108 are provided for the inner shaft 166. The slots 142, 144 are covered by a contoured proximal part 118 of the blade portion 102 so that the interior of the blade portion 102 and the expansion mechanism will not be open to tissue or bodily fluids. Optionally, a contoured distal part 120 of the blade portion 102 may be attached to the second wing 108.

When the adjustment knob 114 is rotated in a first direction, the inner shaft 166 rotates the pinion gear 164, which causes of each of the rack gears 160, 162 to move outward, telescopically sliding the wings 106, 108 apart to increase the width of the blade portion 102, as shown in FIGS. 24 and 25. When the adjustment knob 114 is rotated in the opposite direction, the inner shaft 166 rotates the pinion gear 164 in the opposite direction, which causes of each of the rack gears 160, 162 to move inward, telescopically sliding the wings 106, 108 together to decrease the width of the blade portion 102, as shown in FIGS. 22 and 23. Stops may be provided to prevent the wings 106, 108 from overextending and to keep the rack gears 160, 162 from becoming disengaged from the pinion gear 164.

The embodiment of the intervertebral shaver 100 shown in FIGS. 22-25 also utilizes a very wide rack-and-pinion gearset to assure stable and parallel motion of the wings 106, 108. The same could also be accomplished using a pair of narrower, spaced apart rack-and-pinion gearsets that act in synchrony to assure stable and parallel motion of the wings 106, 108.

In each of the embodiments of the adjustable intervertebral shaver 100 described herein, the various components of the device can be fabricated from metals, such as stainless steel, titanium or cobalt-chromium alloys, or from high-strength polymers or fiber reinforced polymer composites or from a combination of these materials. The adjustable intervertebral shaver 100 may be constructed to be reusable, using materials that will stand up to repeated sterilization and use. Alternatively, the adjustable intervertebral shaver 100 may be constructed for one-time use, using economical materials and construction methods that would allow the device to be disposable. In either case, the device may optionally be packaged sterile and ready for use.

While the present invention has been described herein with respect to the exemplary embodiments and the best mode for practicing the invention, it will be apparent to one of ordinary skill in the art that many modifications, improvements and subcombinations of the various features and embodiments, adaptations and variations can be made to the invention without departing from the spirit and scope thereof.

What is claimed is:

1. An adjustable intervertebral shaver, comprising:
a flat blade portion having a length, a width and a thickness;
a handle portion connected to the blade portion;
an expansion mechanism for selectively adjusting the width of the blade portion;
and an adjustment knob on the handle portion for operating the expansion mechanism.

2. The adjustable intervertebral shaver of claim 1, wherein the handle portion comprises a shaft connected to the blade portion and a T-handle connected to the shaft.

3. The adjustable intervertebral shaver of claim 1, further comprising a first scraping surface on a first lateral edge of the blade portion and a second scraping surface on a second lateral edge of the blade portion.

4. The adjustable intervertebral shaver of claim 1, wherein the blade portion has a fully contracted position and a fully expanded position and wherein the width of the blade portion in the fully expanded position is approximately twice the width of the blade portion in the fully contracted position.

5. The adjustable intervertebral shaver of claim 1, wherein the blade portion comprises a first wing and a second wing; and wherein the expansion mechanism selectively adjusts the width of the blade portion by moving the first wing and the second wing relative to one another.

6. The adjustable intervertebral shaver of claim 1, wherein the blade portion comprises a central housing, a first wing slidably received within the central housing and a second wing slidably received within the central housing;
and wherein the expansion mechanism selectively adjusts the width of the blade portion by extending or retracting the first wing and the second wing relative to the central housing.

7. The adjustable intervertebral shaver of claim 1, wherein the blade portion comprises a first wing and a second wing, the first wing being configured to slide telescopically within the second wing;
and wherein the expansion mechanism selectively adjusts the width of the blade portion by telescopically extending or retracting the first wing and the second wing relative to one another.

8. The adjustable intervertebral shaver of claim 1, wherein the blade portion comprises a first wing and a second wing; wherein the expansion mechanism comprises at least one threaded rod having a first end and a second end, the first end being threaded in the opposite direction from the second end, a first threaded hole in the first wing threaded to engage the first end of the threaded rod, a second threaded hole in the first wing threaded to engage the second end of the threaded rod;
and wherein, when the threaded rod is rotated in a first direction, the first wing and the second wing move apart from one another to increase the width of the blade portion, and, when the threaded rod is rotated in a second direction, the first wing and the second wing move toward one another to decrease the width of the blade portion.

9. The adjustable intervertebral shaver of claim 8, further comprising a pinion gear connected to the threaded rod and a worm gear engaging the pinion gear, the worm gear connected to the adjustment knob by an inner shaft;
wherein, when the adjustment knob is rotated in a first direction, the first wing and the second wing move apart from one another to increase the width of the blade portion and when the adjustment knob is rotated in a second direction, the first wing and the second wing move toward one another to decrease the width of the blade portion.

10. The adjustable intervertebral shaver of claim 1, wherein the blade portion comprises a first wing and a second wing;
wherein the expansion mechanism comprises a first threaded rod having a first end and a second end, and a second threaded rod having a first end and a second end, the first ends of the threaded rods being threaded in the opposite direction from the second ends of the threaded rods, a first pair of threaded holes in the first wing threaded to engage the first ends of the threaded rods, a second pair of threaded holes in the first wing threaded to engage the second ends of the threaded rods, a first pinion gear connected to the first threaded rod, a second pinion gear connected to the second threaded rod and a worm gear engaging the first and second pinion gears, the worm gear being connected to the adjustment knob by an inner shaft;
and wherein, when the adjustment knob is rotated in a first direction, the worm gear rotates the first and second pinion gears, which in turn rotates the threaded rods, causing the first wing and the second wing to move apart from one another to increase the width of the blade portion, and, when the adjustment knob is rotated in a second direction, the worm gear rotates the first and second pinion gears, which in turn rotates the threaded rods, causing the first wing and the second wing to move toward one another to decrease the width of the blade portion.

11. The adjustable intervertebral shaver of claim 1, wherein the blade portion comprises a first wing and a second wing; wherein the expansion mechanism comprises at least one scissor mechanism connected between the first wing and the second wing;
and wherein, when a proximal-to-distal length of the scissor mechanism is decreased, a lateral width of the scissor mechanism increases, causing the first wing and the second wing to move apart from one another to increase the width of the blade portion, and, when the proximal-to-distal length of the scissor mechanism is increased, the lateral width of the scissor mechanism decreases, causing the first wing and the second wing move toward one another to decrease the width of the blade portion.

12. The adjustable intervertebral shaver of claim 11, further comprising a pair of push-pull rods connected to the scissor mechanism to control the proximal-to-distal length of the scissor mechanism.

13. The adjustable intervertebral shaver of claim 1, wherein the blade portion comprises a first wing and a second wing;
wherein the expansion mechanism comprises a first scissor mechanism and a second scissor mechanism connected between the first wing and the second wing, and a pair of push-pull rods connected to the scissor mechanism to control a proximal-to-distal length of the first scissor mechanism and the second scissor mechanism;
and wherein, when the proximal-to-distal length of the first scissor mechanism and the second scissor mechanism is decreased, a lateral width of the first scissor mechanism and the second scissor mechanism increases, causing the first wing and the second wing to move apart from one another to increase the width of the blade portion, and, when the proximal-to-distal length of the first scissor mechanism and the second scissor mechanism is increased, the lateral width of the first scissor mechanism and the second scissor mechanism decreases, causing the first wing and the second wing move toward one another to decrease the width of the blade portion.

14. The adjustable intervertebral shaver of claim 1, wherein the blade portion comprises a first wing and a second wing; wherein the expansion mechanism comprises a first rack gear connected to the first wing and a second rack gear connected to the second wing, a pinion gear that engages the first rack gear and the second rack gear; and wherein, when the pinion gear is rotated in a first direction, the first rack gear and the second rack gear move outward, causing the first wing and the second wing to move apart from one another to increase the width of the blade portion, and, when the pinion gear is rotated in a second direction, the first rack gear and the second rack gear move inward, causing the first wing and the second wing to move toward one another to decrease the width of the blade portion.

15. The adjustable intervertebral shaver of claim 14, further comprising an inner shaft connecting the pinion gear to the adjustment knob; wherein, when the adjustment knob is rotated in a first direction, the first wing and the second wing move apart from one another to increase the width of the blade portion and when the adjustment knob is rotated in a second direction, the first wing and the second wing move toward one another to decrease the width of the blade portion.

16. A method of surgically preparing an intervertebral space, comprising:
inserting a blade portion of an adjustable intervertebral shaver into the intervertebral space;
adjusting a width of the blade portion of the adjustable intervertebral shaver while the blade portion is within the intervertebral space;
and manipulating the blade portion to break up or remove intervertebral disc material from the intervertebral space.

17. The method of claim 16, wherein the width of the blade portion of the adjustable intervertebral shaver is adjusted using an adjustment knob located on a handle portion of the adjustable intervertebral shaver outside of the intervertebral space while the blade portion is within the intervertebral space.

18. The method of claim 16, further comprising:
adjusting the adjustable intervertebral shaver to increase the width of the blade portion while the blade portion is within the intervertebral space;
and manipulating the blade portion to break up or remove additional intervertebral disc material from the intervertebral space.

19. The method of claim 16, further comprising:
adjusting the width of the blade portion to a fully contracted position;
and removing the blade portion of the adjustable intervertebral shaver from the intervertebral space.
